(19) United States
(12) Patent Application Publication
(10) Pub. No.: US 2014/0294958 A1
(43) Pub. Date: Oct. 2, 2014

(54) LUBRICIOUS COATINGS

(71) Applicant: DSM IP ASSETS B.V., Heerlen (NL)

(72) Inventors: Johannes Wilhelmus Belt, Echt (NL); Yogesh Nathalal Gandhi, Echt (NL)

(21) Appl. No.: 14/354,055

(22) PCT Filed: Nov. 9, 2012
(86) PCT No.: PCT/EP2012/072210

§ 371 Date: Apr. 24, 2014

(30) Foreign Application Priority Data
Nov. 10, 2011 (EP) 11188598.4

(51) Int. Cl. A61K 9/28

(52) U.S. Cl. CPC A61K 9/286; A61K 9/284; A61K 9/2893
USPC 424/475; 514/772.5; 427/2.14

(57) ABSTRACT

The present invention relates to lubricious coatings comprising an ionomer and a hydrophilic polymer wherein the ionomer is cross-linked using a suitable cross-linking agent, and optionally also conventional excipients, such as a surfactant, a plasticizer and/or a filler. The invention also relates to pharmaceutical products covered by such lubricious coatings, compositions for preparing these lubricious coatings and a method for providing pharmaceutical products with these lubricious coatings.

The present invention relates to lubricious coatings, pharmaceutical products covered by such lubricious coatings, lubricious coating compositions and a method for the coating of pharmaceutical products.

Lubricious coatings are known for example from U.S. Pat. No. 7,547,474, WO 2009/135067 and WO 2010/059530.

U.S. Pat. No. 7,547,474 describes a lubricious coating formed of an interpenetrating polymer network comprising a hydrophilic polymer (such as polyalkylene glycols, more in particular poly(ethylene oxide) entrapped on the surface of a substrate, and a cross-linked polymer (such as poly(met)acrylates). This publication lacks any objective proof for an improvement of the lubricity by the application of the coating described. Many of the chemical components of this coating may be undesirable for pharmaceutical applications. Furthermore, the method of polymerization of the coating may cause interaction with the pharmaceutical substance to be coated.

WO 2009/135067 discloses lubricious coatings comprising a film forming agent (which is a hydrophilic polymer exemplified by e.g. hydroxypropylcellulose) and a coating agent which is an apolar substance (exemplified by Carnuba wax, various stearates, silicon dioxide or talc). The coating agent described is a hydrophobic material which will retard or even prevent the uptake of water which is necessary for the coatings to become slippery. For that reason hydrophobic materials in the coating formulation are undesired. Also there is no indication given that these coatings become more slippery when wetted sufficiently.

According to WO 2010/059530 lubricious coatings can be prepared from a hydrophilic polymer (such as poly(vinyl pyrrolidone) and a natural product derived directly from plants or animals (like shellac). Also this publication lacks any objective proof for an improvement of the lubricity by the application of the coating described.

As described above these prior lubricious coatings have the following shortcomings (1) the coating contains a hydrophobic material and (2) the polymerization process is a chemical process with a considerable risk that of interacting with the pharmaceutical substance to be coated.

Lubricious coatings according to the present invention can solve these shortcomings.

A lubricious coating according to the present invention comprises a combination of an ionomer and a hydrophilic polymer wherein the ionomer is cross-linked using a cross-linking agent capable of cross-linking the ionomer.

As used herein, with an ionomer is meant a polymer bearing ionizable or ionic functionalities such as carboxylic acid, quaternary ammonium salts, ammonium salts, carboxylate salts or sulfonate salts. Examples are polyacrylic acid, polyacrylic acid sodium salt, polysaccharides such as alginic acid, sodium alginate, kappa carrageenan, lambda carrageenan, pectin, sodium carboxymethylcellulose, sodium hyaluronate, copolymers of acrylamide and acrylic acid or methacrylic acid, copolymers of ethylene and acrylic or methacrylic acid, phosphatespoly(acrylamide-co-dialkylammoniumchloride) or poly(methacrylamide-co-dialkylammoniumchloride).

The Molecular weight (Mw) of the ionomers can be between 1000 and 10.000.000 Dalton, preferably between 20.000 and 2.000.000 Dalton.

As used herein, with a hydrophilic polymer is meant a polymer that dissolves in water. the hydrophilic polymer may be selected from the group consisting of polyethers, polyurethanes, polyamides, polyoxazolines, polypeptides, or polysaccharides. Examples are polyvinyl pyrolidone (PVP), polyvinyl caprolactam, polyethylene glycol (PEG), polyacrylamide, polyvinylalcohol (PVA), gelatin, agar, chitosan, hydroxypropyl cellulose, hydroxyethyl cellulose, and starch. The Molecular weight (Mw) of the hydrophilic polymers can be between 1000 and 10.000.000 Dalton, preferably between 20.000 and 2.000.000 Dalton.

As used herein, with a cross-linking agent is meant an agent which has the ability to cross-link the ionomer used according to the invention via ionic interaction. Examples are soluble Ca²⁺ salts (which can for example cross-link sodium alginate), soluble K⁺ salts (which can cross-link κ-caragenan), poly cationic compounds which can crosslink for example negatively charged ionomers, polyanionic compounds which can for examples crosslink positively charged ionomers, polyamine compounds that can crosslink polymers bearing acidic functionalities, polycarboxylic acid compounds that can crosslink polymers bearing amine functionalities.

In a further embodiment the lubricious coating according to the present invention comprises 10-80% (w/w) of the ionomer and 20-90% (w/w) of the hydrophilic polymer.

In a further embodiment the lubricious coating according to the present invention comprises 0.1-10% (w/w) of the cross-linking agent.

In a particular embodiment the ionomer may be alginate.

In a further embodiment the lubricious coating according to the present invention comprises as an ionomer alginate and as the cross-linking agent capable of cross-linking the ionomer it comprises a calcium salt, preferably CaCl₂.

In a further particular embodiment the hydrophilic polymer may be PVP

In a further particular embodiment the alginate and the PVP may be present in the coating in a weight ratio of about 1:2.

In a further embodiment the lubricious coating according to the present invention further comprises as an ingredient a surfactant.

In a further embodiment the lubricious coating according to the present invention comprises 0.1-5% (w/w) of the surfactant.

As used herein, with a surfactant is meant a water-soluble surface-active agent comprised of a hydrophobic portion, usually a long alkyl chain, attached to hydrophilic or water solubility enhancing functional groups. Surfactants can be categorized according to the charge present in the hydrophilic portion of the molecule (after dissociation in aqueous solution): ionic surfactants, for example anionic or cationic surfactants, and non-ionic surfactants. Examples of ionic surfactants include Sodium dodecylsulfate (SDS), Sodium cholate, Bis(2-ethylhexyl)sulfosuccinate Sodium salt, Cetyltrimethylammoniurnbromide (CTAB), Lauryldimethylamine-oxide (LDAO), N-Lauroylsarcosine Sodium salt and Sodium deoxycholate (DOC). Examples of non-ionic surfactants include Alkyl Polyglucosides such as TRITON™ BG-10 Surfactant and TRITON CG-110 Surfactant and Tween (such as Tween 20 and Tween 80), Branched Secondary Alcohol Ethoxylates such as TERGITOL™ TMN Series, Ethylene Oxide/Propylene Oxide Copolymers, such as TERGITOL L Series, and TERGITOL XD, XH, and XJ Surfactants, Nonylphenol Ethoxylates such as TERGITOL NP Series, Octylphenol Ethoxylates, such as TRITON X Series, Secondary Alcohol Ethoxylates, such as TERGITOL 15-S Series and Specialty Alkoxylates, such as TRITON CA Surfactant, TRITON N-57 Surfactant, TRITON X-207. Also a mixture of these surfactants can be used.

In a further embodiment the lubricious coating according to the present invention further comprises as an ingredient a plasticizer.

In a further embodiment the lubricious coating according to the present invention comprises from about 0.01 wt % to about 50 wt %, preferably from about 1 wt % to about 5.0 wt %, of the plasticizer based on the total weight of the dry coating.

As used herein, with a plasticizer is meant an agent that can enhance the flexibility of the coating. Said plasticizing agent may be included in the hydrophilic coating formulation in a concentration of from about 0.01 wt % to about 50 wt % based on the total weight of the dry coating, preferably from about 1 wt % to about 5.0 wt %. Suitable plasticizers are high boiling compounds, preferably with a boiling point at atmospheric pressure of >200° C., and with a tendency to remain homogeneously dissolved and/or dispersed in the coating. Examples of suitable plasticizers are mono- and polyalcohols and polyethers, such as decanol, glycerol, ethylene glycol, diethylene glycol, polyethylene glycol and/or copolymers with propylene glycol and/or fatty acids. Also a mixture of plasticizers can be used.

In a further embodiment the lubricious coating according to the present invention further comprises as an ingredient a filler.

In a further embodiment the lubricious coating according to the present invention comprises the filler in an amount of 0.1 up to 10 times the weight of the ionomer and hydrophilic polymer.

As used herein, with a filler is meant an agent that is insoluble in the solvent of the coating formulation and that prevents sticking of the tablets during the coating operation and improves the integrity of the coating. Suitable examples of filler are talc, calcium carbonate and magnesium carbonate. Also a mixture of these fillers can be used. Particle size can be from about 50-300 mesh.

In a further embodiment the lubricious coating according to the present invention comprises as an ingredient an anti tacking agent (such as talc), a pigment (such as a dye, aluminum black or ferric oxide) and/or an opacifying agent (such as titanium oxide).

WO02098393 discloses tablets which are coated with a mixture of sodium alginate and PVP-VA-copolymer. The alginate is, however, not been cross-linked.

WO10059530 discloses pharmaceutical articles having a lubricious coating comprising a hydrophilic polymer and a natural product derived directly from plants or animals. Examples of the natural product mentioned are gums, which are defined as polysaccharides of natural origin, such as carrageenan. Sodium alginate was not specifically mentioned and, furthermore, no mention was made of the possible cross-linking of the natural product.

WO0132150 discloses coating compositions comprising microcrystalline cellulose, carrageenan and a so-called strengthening polymer and/or plasticizer. As a strengthening polymer according to this publication may be used hydroxyethylcellulose, HPMC, hydroxypropylcellulose, ethylcellulose, methylcellulose and polyvinylpyrrolidone (PVP). Suitable plasticizers according to this publication include polyethylene glycol, advantageously a high molecular weight polyethylene glycol, triacetin, dibutyl sebacate, propylene glycol, sorbitol, glycerin, and triethyl citrate. However, the description does not mention the possibility of cross-linking of the carrageenan in the coating.

U.S. Pat. No. 6,274,162 discloses a dry film coating for pharmaceuticals, food, confectionary forms, agricultural seeds, etc., which comprises gelatin and/or hydroxyethyl cellulose, and at least one of the following components: a secondary film former, a plasticizer, a glidant, a suspension aid, a colorant and a flavorant. As an example of the secondary film former sodium alginate is mentioned (as well as glycol alginate). Glycol alginate is also mentioned as a suspension aid. However, no mention is made in this document of the possible cross-linking of alginate in the coating.

The cross-linked coatings according to the present invention have the advantages of better stability as a coating of a pharmaceutical product, a better lubricity, less interaction with the pharmaceutical substance in a thus coated pharmaceutical product and lending to the pharmaceutical product the characteristic that it can be swallowed better, even when the pharmaceutical product is taken with little or no water.

In a further aspect, the present invention relates to coating compositions from which these lubricious coatings can be obtained.

In one embodiment this coating composition comprises a combination of an ionomer, a hydrophilic polymer and a cross-linking agent capable of cross-linking the ionomer, as well as a suitable solvent.

In a further embodiment the coating composition according to the present invention comprises 0.1-10% (w/w) of the ionomer and 0.1-20% (w/w) of the hydrophilic polymer and 75-99.5% (w/w) of the solvent.

In a particular embodiment the ionomer may be alginate.

In a further particular embodiment the hydrophilic polymer may be PVP.

In a further particular embodiment the alginate and the PVP may be present in the coating in a weight ratio of about 1:2.

In a further embodiment the coating composition according to the present invention further comprises as an ingredient a surfactant.

In a further embodiment the coating composition according to the present invention comprises 0.0001-1% (w/w) of the surfactant.

In a further embodiment the coating composition according to the present invention further comprises as an ingredient a plasticizer.

In a further embodiment the coating composition according to the present invention comprises 0.01-5% (w/w) of the plasticizer.

In a further embodiment the coating composition according to the present invention further comprises as an ingredient a filler.

In a further embodiment the coating composition according to the present invention comprises 0.5-25% (w/w) of the filler.

In a further embodiment the coating composition according to the present invention comprises 0.01-1% (w/w) of the cross-linking agent.

In a further embodiment the coating composition according the present invention comprises a mixture of ionomers or a mixture of different molecular weights versions of a given ionomer

In a further embodiment the coating composition according the present invention comprises a mixture of hydrophilic polymers or a mixture of different molecular weights versions of a given hydrophilic polymer

In a further aspect the present invention relates to a coated pharmaceutical product comprising a pharmaceutical product which is at the exterior surface is being covered by a lubricious coating substantially as described herein.

As used herein, with a pharmaceutical product is meant a formulated solid pharmaceutical composition e.g. in the form of a pill, tablet, capsule.

This pharmaceutical product may additionally be provided with a functional coating layer, such as a layer to protect the pharmaceutical product or to prevent the product to dissolve in the stomach, or such as a layer to extend the release of the active ingredient.

In a further aspect, the present invention relates to coated pharmaceutical products having a coating comprising a combination of an ionomer, a hydrophilic polymer, wherein the ionomer is cross-linked with a suitable cross-linking agent and optionally at least one component selected from the group consisting of a surfactant, a plasticizer, a filler, an anti-tacking agent, a pigment and/or an opacifying agent.

In a further aspect, the present invention relates to a method for the coating of pharmaceutical products, wherein a coating composition according to the present invention is being applied to a pharmaceutical product.

The film coating of polymer over the tablets can for example be achieved using a pan coating.

In pan coating, tablets are tumbled in a perforated stainless steel coating pan positioned at an angle (e.g. of approximately 45 degrees) to the horizontal surface at certain speed. Polymers and other ingredients are dissolved and/or suspended in purified water or other suitable solvent. Coating suspension can be sprayed using a pump via nozzle attached to compressed air. Hot air can be blown through the coater at same time that dries the liquid forming a dried film over the tablet. After a pre-determined amount of spray suspension is applied, tablets can be dried further with hot air to remove any trace amount of water in the tablets.

The uniformity and precision of coating can be controlled by maintaining load size, air flow, air temperature, and spray rate of the suspension, atomizing air pressure, and weight gain of the tablet.

In a further aspect, the present invention relates to a method for coating of a pharmaceutical product, wherein to the exterior surface of the pharmaceutical product is first applied a lubricious coating composition comprising an ionomer and a hydrophilic polymer and optionally one or more members of the group consisting of a surfactant, a plasticizer and a filler, and hereafter is applied an aqueous composition comprising a cross-linking agent capable of cross-linking the ionomer.

In a further aspect, the present invention relates to a method for coating of a pharmaceutical product, wherein to the exterior surface of the pharmaceutical product is applied a lubricious coating composition comprising an ionomer, a hydrophilic polymer and a cross-linking agent capable of cross-linking the ionomer, and optionally one or more members of the group consisting of a surfactant, a plasticizer and a filler.

### THE COMPOSITION OF PLACEBO TABLETS AND THE WAY THEY WERE PRODUCED

### A. Composition

TABLE 1
Composition of placebo tablets

| Ingredients | % w/w | mg/tablet |
|---|---|---|
| Micro Cell, NF/EP/JP (Avicel PH102) | 99.00 | 594.0 |
| Magnesium Stearate, NF | 1.00 | 6.0 |
| Total | 100.00 | 600.0 |

### B. Manufacturing Method

Microcrystalline cellulose and Magnesium Stearate were dispensed per batch record. Both the ingredients were blended for 5 minutes in a twin-shell blender. The tablets were compressed using a rotary tablet press. The average tablets weight was maintained between 570-630 mg range. Average hardness of the tablets was maintained in range of 9-13 kp. To avoid any breakage of tablets during coating, friability was kept under 1%.

### EXAMPLE 1

### Coating Process With Coating Composition 1

A. Composition

TABLE 2
Coating composition 1

| | | |
|---|---|---|
| Ingredients | % w/w | Formula Weight (g) |

Core Tablets

| | |
|---|---|
| Placebo Tablets, 600 mg | 1000.00 |

Coating Suspension

| | | |
|---|---|---|
| PVP K90 | 1.33 | 14.00 |
| Sodium Alginate | 0.85 | 9.00 |
| Glycerol | 0.06 | 0.60 |
| Tween 80 | 0.01 | 0.15 |
| Talc | 5.00 | 52.83 |
| Purified Water (Portion 1) | 92.75 | 980.00 |
| Total | 100.00 | 1056.58 |

Post-Coating Solution

| | | |
|---|---|---|
| Calcium Chloride Dihydrate | 0.50 | 2.50 |
| Purified Water (Portion 2) | 99.50 | 497.50 |
| Total | 100.00 | 500.00 |

B. Preparation Coating Suspension:

In a suitable tank equipped with a mixer, Purified Water (Portion 1) was added. Sodium Alginate was then added slowly to Purified Water and mixed until the solution was clear. Then Glycerol was added to the same solution, followed by Tween 80. Mixing was continued until the solution was homogenous and clear. Thereafter, PVP K90 was added slowly to the solution and mixed until the resulting solution was clear. Talc was added to the solution. Mixing was continued for at least 10 minutes after addition of Talc, to ensure that Talc suspended well in the solution.

C. Coating Process:

Tablets were placed into a 12″ perforated coating pan. The tablet bed was warmed, at an exhaust air temperature of about 40° C. Once the exhaust temperature reached to about 40° C., the spraying was started at rate of about 3.5 g/min. Other parameters such as Inlet Air Flow (CFM), Exhaust Air Temperature (° C.), Inlet Temperature (° C.), Pan Speed (rpm) and Atomization Air Pressure (psi) were also adjusted as needed. The coating process was continued until a weight gain of approximately 5% was achieved.

D. Post-Coating Solution Preparation:

In another suitable container, Purified Water (Portion 2) was added. To the Purified Water, Calcium Chloride Dihydrate was added and mixed until Calcium Chloride Dihydrate was completely soluble

E. Post-Coating Process:

The Calcium Chloride Dihydrate 0.5% w/v solution was applied until 1, 5 OR 10% saturation on a molar base of sodium alginate was achieved on the tablets. The amount of post-coating solution to be sprayed was determined by performing the calculations and the amount of solution needed was consumed accordingly.

### EXAMPLE 2

### Coating Process with Coating Composition 2

A. Composition

TABLE 3
Coating composition 2

| | | |
|---|---|---|
| Ingredients | % w/w | Formula Weight (g) |

Core Tablets

| | |
|---|---|
| Placebo Tablets, 600 mg | 1000.00 |

Coating Suspension

| | | |
|---|---|---|
| Povidone K90 | 1.28 | 14.00 |
| Povidone K27-33 | 2.28 | 25.00 |
| Sodium Alginate | 0.82 | 9.00 |
| Glycerol | 0.06 | 0.60 |
| Tween 80 | 0.01 | 0.15 |
| Talc | 6.00 | 65.66 |
| Purified Water (Portion 1) | 89.55 | 980.00 |
| Total | 100.00 | 1094.41 |

Post-Coating Solution

| | | |
|---|---|---|
| Calcium Chloride Dihydrate | 0.50 | 2.50 |
| Purified Water (Portion 2) | 99.50 | 497.50 |
| Total | 100.00 | 500.00 |

The coating suspension manufacturing and coating process were similar as described in Example 1. Only change was made in composition of the suspension and in the degree of saturation (10 and 25%).

### EXAMPLE 3

### Testing the Lubricity or Slipperiness

A. Experimental

The lubricity or slipperiness of tablets was measured according a sled-test with the following test set-up.

The sled consisted out of a polycarbonate plate with a cavity in each corner for positioning the tablets. The dimensions of the cavities are constructed in such a way that the tablets exactly fit in and the tablets stick out in such a way that the sled rests on the tablets. Dimensions of the sled were (length)×(width)×(height)=100×100×8 mm. The weight of the sled is 100 grams.

A water bath was provided with a glass plate with just enough water present to cover the glass plate. The water bath is equipped with a pulley. The dimensions of the water bath are (length)×(width)×(heighth)=250×150×8 mm. An exact fitting glass plate with a height of 2 mm was positioned on the bottom of the bath.

The sled with the tablets is positioned on the wet glass plate and connected to the load cel of a Harland FTS 5000 friction tester via the pulley with a fishing line. The pulley is positioned in such a way that the line is horizontally oriented between the connector on the sled and the pulley. The line is vertically oriented between the pulley and the load cell. On top of the sled a weight of 150 grams is positioned

The friction measurement is started by activating the FTS which pulls the sled bearing the tablets over the glass plate for a distance of 12 cm with a speed of 1 cm/s during which the friction is recorded. The average friction over a distance of 8 cm is recorded. An average of 5 experiments was performed.

B. Results

The results of the friction test in Table 3 indicate the significant improvement in friction reduction that can be achieved with the invented coatings described in this patent application.

The placebo tablets had high friction values of 180 gram while the coated tablets all had friction values that were significantly lower.

TABLE 4
Results of friction tests

| | | Saturation with | |
|---|---|---|---|
| | | CaCl2 | Friction |
| Exp | Composition | (%) | (gram) |
| 1 | Uncoated tablet | — | 191 |
| 2 | Example 1 | 1 | 64 |
| | | 5 | 68 |
| | | 10 | 94 |
| 3 | Example 2 | 10 | 71 |
| | | 25 | 78 |

### EXAMPLE 4

### Metformin Tablet Formulation Coated with Coating Composition 2

A. Composition of Metformin Tablets

TABLE 5
Metformin Tablet Formulation

| Ingredients | % w/w | mg/tablet |
|---|---|---|
| Metformin HCl | 83.33 | 500.0 |
| Micro Cell, NF/EP/JP | 5.50 | 33.0 |
| Copovidone | 7.00 | 42.0 |
| Hydroxy Propyl Methcel, USP | 0.50 | 3.0 |
| Crospovidone, NF/EP/JP | 3.00 | 18.0 |
| Talc, USP | 0.17 | 1.0 |
| Magnesium Stearate, NF | 0.50 | 3.0 |
| Purified Water, USP/EP* | | |
| Total | 100.00 | 600.0 |

*Removed during drying.

Micro Cell is Microcrystalline Cellulose (Avicel)

Copovidone is Kollidon VA 64

Hydroxy Propyl Methcel is Hydroxy Propyl Methyl Cellulose (HPMC)

Crospovidone is Polyplasdone XL

The preparation of the coating composition, the coating process, the post-coating solution preparation and the post-coating process were similar as described in Example 1 using the compositions as summarized in table 6.

TABLE 6
Coated Metformin Tablet Formulation

| | | |
|---|---|---|
| Ingredients | % w/w | Formula Weight |

Core Tablets

| | | |
|---|---|---|
| Metformin Tablets, 500 mg | 9.000 | kg |

Coating Suspension

| | | | |
|---|---|---|---|
| Povidone K90 | 1.28 | 64.00 | g |
| Povidone K27-33 | 2.28 | 114.0 | g |
| Sodium Alginate | 0.82 | 41.00 | g |
| Glycerol | 0.06 | 3.00 | g |
| Tween 80 | 0.01 | 0.50 | g |
| Talc | 6.00 | 300.0 | g |
| Purified Water (Portion 1) | 89.55 | 4.48 | kg * |
| Total | 100.00 | 5.00 | kg |

Post-Coating Solution

| | | | |
|---|---|---|---|
| Calcium Chloride Dihydrate | 0.50 | 5.00 | g |
| Purified Water (Portion 2) | 99.50 | 995.00 | g |
| Total | 100.00 | 1000.00 | g |

Accelerated stability (40C/75% RH) performed on both coated and uncoated tablets through 2 months. Analytical testing performed on both tablets showed that the presence of the coating did not impact any of the critical quality attributes of the Metformin Tablets measured as defined in USP monograph for Metformin Hydrochloride Tablets with 500 mg label claim.

The lubriciousness of the Metformin tablets according to the present invention was greatly improved as compared to the uncoated tablets.

1. Lubricious coating comprising an ionomer and a hydrophilic polymer wherein the ionomer is cross-linked using a cross-linking agent capable of cross-linking the ionomer.

2. Lubricious coating according to claim 1, further comprising as an ingredient a surfactant.

3. Lubricious coating according to claim 1, further comprising as an ingredient a plasticizer.

4. Lubricious coating according to claim 1, further comprising as an ingredient a filler.

5. Lubricious coating according to claim 1 the ionomer is alginate.

6. Lubricious coating according to claim 5, wherein the cross-linking agent cross-linking the ionomer is a calcium salt.

7. Lubricious coating according to claim 1, wherein the hydrophilic polymer is PVP.

8. Lubricious coating according to claim 6, wherein alginate and PVP are present in a weight ratio of about 1:2.

9. Lubricious coating composition comprising the ingredients of a lubricious coating according to claim 1 as well as an aqueous solvent.

10. Coated pharmaceutical product comprising a pharmaceutical product which is at the exterior surface is being covered by a lubricious coating according to claim 1.

11. Process for coating of a pharmaceutical product, wherein to the exterior surface of the pharmaceutical product is first applied a lubricious coating composition comprising an ionomer and a hydrophilic polymer and optionally one or more members of the group consisting of a surfactant, a plasticizer and a filler, and whereafter is applied an aqueous composition comprising a cross-linking agent capable of cross-linking the ionomer.

12. Process for coating of a pharmaceutical product, wherein to the exterior surface of the pharmaceutical product is applied a lubricious coating composition comprising an ionomer, a hydrophilic polymer and a cross-linking agent capable of cross-linking the ionomer, and optionally one or more members of the group consisting of a surfactant, a plasticizer and a filler.
